(19) United States
(12) Plant Patent Application Publication
(10) Pub. No.: US 2002/0073450 P1
(43) Pub. Date: Jun. 13, 2002

(54) Miniature rose plant

(76) Inventor: George Mander, Coquitlam (CA)

Correspondence Address: MICHAEL C. WILLIAMS dba, THE MINE ROSE GARDEN., PO BOX 203, Cross Hill, SC, 29332, US

(21) Appl. No.: 09/730,961

(22) Filed: Dec. 7, 2000

(51) Int. Cl. A01H 5/00

(52) U.S. Cl. PLT/116

(57) ABSTRACT

This new and distinct variety of miniature rose plant is characterized by blooms of salmon and medium yellow coloration, borne on an upright, vigorous plant with slightly glossy greenish-purple new foliage and slightly glossy green old foliage. This plant is easily propagated from cuttings, and its blooms are long lasting on the plant and as cut flowers.

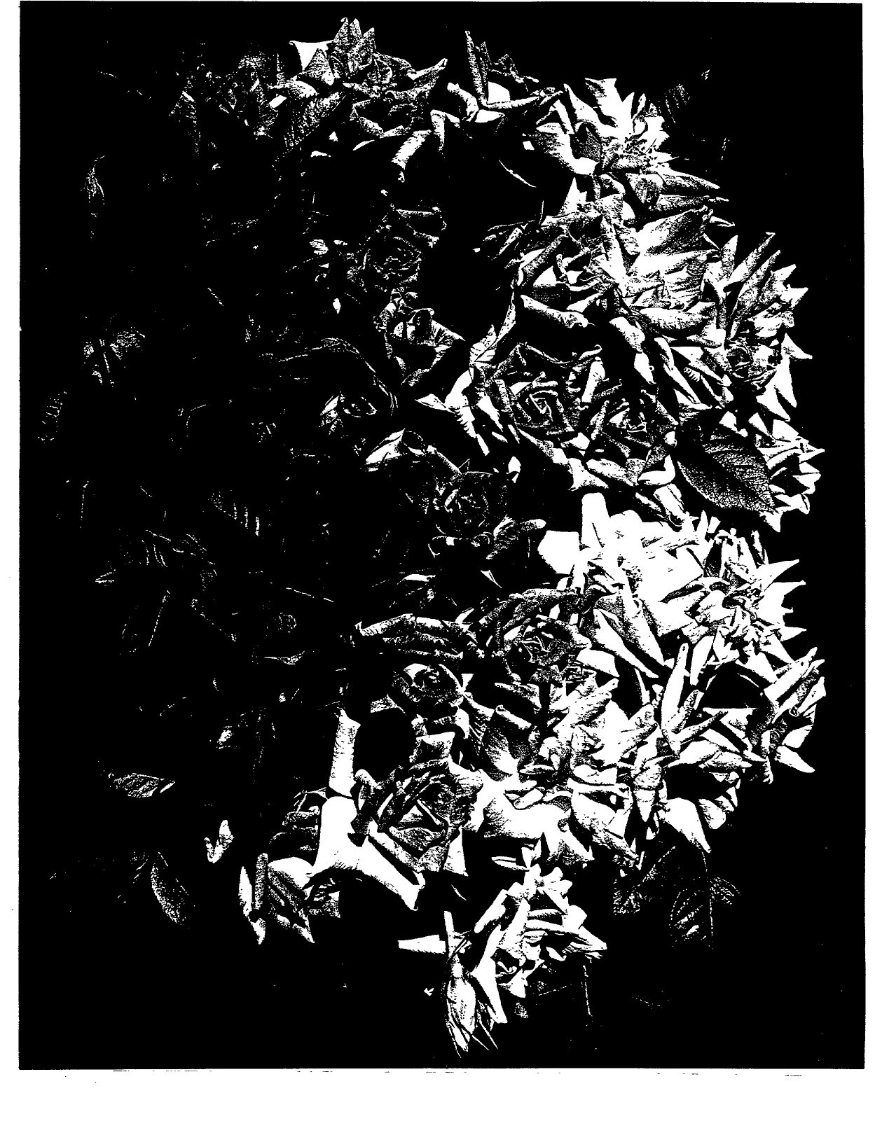

The accompanying photograph shows typical specimens of the vegetative growth and flowers of the new variety in different stages of development and as depicted in color as nearly true as it is reasonably possible to make the same in a color photograph of this character.

### DETAILED BOTANICAL DESCRIPTION OF NEW VARIETY

The following is a detailed description of the new rose variety, with color terminology in accordance with the plate numbers of The Royal Horticultural Society Colour Chart except where common dictionary terms of color definitions are employed.

Breeding: Sport of Glowing Amber

Class: Miniature rose

Variety: Manstar

Blooming Habit: Recurrent

The following observations were made from specimens grown outdoors in Coquidam, British Columbia, Canada in August, 2000.

Flower:

Bud.—(1) Size: Circumference: 1¼″ to 1⅜″ Diameter: ⅜″ (2) Form: pear-shaped (3) Color: When sepals first divide: 30A at peak to 12B at base When petals begin to unfurl: 32B at peak to 12B at base When half-blown: inside of petals: 30C at peak to 12B at base reverse side of petals: 12B (4) Sepals: As long as the bud, thin and pointed. (5) Peduncle: Length: 1 inch Aspect: smooth Strength: erect Color: 139C.

Bloom.—(1) Size: Average size when fully expanded: 2 inches (2) Borne: singly, on occasion with two to three buds (3) Form: high centered at first, petals loosely quilled outward at maturity (4) Petalage: Number of petals under normal conditions: 35 (5) Color: (during first two days) Inner surface of petals at center of flower: 32C Inner surface of outer petals: 28C blended with 21C Base of petals (Point of attachment): 9B Underside Petal reverse: 9B General tonality from a distance: 30D blended with 21C (6) Color Change: As bloom ages: Orange changes to salmon in sunny weather. General tonality: 32C with 9C at base After three or more days: Salmon becomes lighter and changes towards yellow.

Petals.—(1) Texture: thick (2) Appearance: inside-velvety, outside-satiny (3) Form: high-centered (4) Arrangement: imbricated (5) Petaloids in center: few (6) Persistence: do not drop off cleanly (7) Fragrance: none (8) Lasting Quality: on the plant-3 days, as a cut flower-4 days.

Reproductive organs:

Stamens, filaments and anthers.—(1) Arrangement: circularly arranged around numerous styles, slightly shorter than styles. (2) Color: 15A.

Pollen.—Color: 24A.

Styles.—even, medium length, bunched.

Stigmas.—Color: 150C.

Hips: none.

Plant:

Form.—upright.

Growth.—(1) Description: very vigorous, uniform branching (2) Height attained: about 30 inches.

Foliage.—compound of 5 to 7 leaflets (1) Size: medium (2) Quantity: abundant (3) Color: New foliage: Upper side: 138A Under side: 138B Old foliage: Upper side: 137A Under side: 137C (4) Shape: oval pointed (5) Texture: Upper side: smooth Under side: veins protrude (6) Edge: serrated (7) Serration: uninformed indentations (8) Leaf Stem: Color: Upper side: 137B Under side: 139C (9) Stipules: short, not bearded (10) Resistance to Disease: Blackspot: good Mildew: good Rust: good.

Wood.—(1) New Wood: Color: 138B Bark Texture: smooth (2) Old Wood: Color: 137C Bark Texture: smooth.

Thorns.—(1) Thorns (prickles): Quantity: On main basals/stems: few On basal/stem laterals: 2-4 each internode Form: flat base, angled down very slightly Length: ¼ inch Color (when young): 177C Position: irregular (2) Prickles: Quantity: On petioles; generally 3 to 4 On underside of leaflet veins: generally none.

Winter hardness.—Protection is needed below 10 degrees Fahrenheit; mounding canes with soil or mulch should provide the needed protection.

I claim:

1. A new and distinct variety of rose plant of the miniature class, substantially as herein shown and described, characterized particularly by blooms of salmon and medium yellow coloration and borne on an upright and vigorous plant with slightly glossy greenish-purple new foliage and slightly glossy green old foliage, said plant being easy to propagate from cuttings, and said blooms being long lasting on the plant and as cut flowers.
